(19) United States
(12) Patent Application Publication
(10) Pub. No.: US 2024/0180131 A1
(43) Pub. Date: Jun. 6, 2024

(54) MEDIUM FOR BREEDING HERMETIA ILLUCENS LARVA THAT PRODUCES HIGHLY ACTIVE ANTIMICROBIAL PEPTIDE (AMP), BREEDING METHOD, AND EXTRACTION METHOD OF AMP

(71) Applicant: Agricultural Products Processing Research Institute, CATAS, Guangdong (CN)

(72) Inventors: Fei WANG, Guangdong (CN); Hui WANG, Guangdong (CN); Qihui ZHANG, Guangdong (CN); Yuliang CHEN, Guangdong (CN); Chaozhong ZHENG, Guangdong (CN); Xiaofang WANG, Guangdong (CN); Leiyu WANG, Guangdong (CN); Jia WU, Guangdong (CN)

(21) Appl. No.: 18/220,461

(22) Filed: Jul. 11, 2023

(30) Foreign Application Priority Data
Dec. 1, 2022 (CN) 202211531619.6

(51) Int. Cl. A01K 67/033; A23K 10/16; A23K 10/30; A23K 50/90

(52) U.S. Cl. CPC A01K 67/033; A23K 10/16; A23K 10/30; A23K 50/90

(57) ABSTRACT

The present disclosure belongs to the technical field of antimicrobial peptide (AMP) production, and relates to a medium for breeding a Hermetia illucens larva that produces a highly active antimicrobial peptide (AMP), a breeding method, and an extraction method of an AMP. The present disclosure provides a medium for breeding a Hermetia illucens larva that produces a highly active AMP, including wheat bran, where the medium has a moisture content of 65% to 70%. In the present disclosure, the medium can realize the rapid and effective production of the highly active AMP by the Hermetia illucens larva.

### CROSS REFERENCE TO RELATED APPLICATION

This patent application claims the benefit and priority of Chinese Patent Application No. 202211531619.6, filed with the China National Intellectual Property Administration on Dec. 1, 2022, the disclosure of which is incorporated by reference herein in its entirety as part of the present application.

### TECHNICAL FIELD

The present disclosure belongs to the technical field of antimicrobial peptide (AMP) production, and specifically relates to a medium for breeding a Hermetia illucens larva that produces a highly active antimicrobial peptide (AMP), a breeding method, and an extraction method of an AMP.

### BACKGROUND

The abuse of antibiotics leads to the enhancement of drug resistance in pathogenic microorganisms, and even promotes the birth of “super pathogens”, thereby weakening an effect of the antibiotics. At present, antibiotic resistance and safety have been listed by the World Health Organization (WHO) as one of the threats to global public health. Since 2015, China has issued relevant documents to prohibit the use of some antibiotics in food animals. Until 2020, the Ministry of Agriculture and Rural Affairs has made it clear that enterprises should stop producing commercial feed containing growth-promoting antibiotics. Therefore, the situation of “prohibiting the use of antibiotics in feed” has become extremely clear. Especially under the background that the use of antibiotics is increasingly strictly restricted, it is an urgent need for the industry to find green, safe, and efficient alternatives to the antibiotics.

Insect-derived antimicrobial peptides (AMPs) are considered to be one of the potential alternatives to antibiotics due to desirable antimicrobial activity, stability, and safety. As a representative of the defense mechanism of insect hosts, AMPs widely exist in many insects and are the main components of a natural immune system of insect hosts. Insect-derived AMPs are mainly composed of small-molecular secondary metabolites as well as small proteins and peptides. Such substances have defense mechanisms against pathogens such as bacteria, fungi, and viruses, and show a great potential as the “antibiotic substitutes”.

The first insect-derived AMP was discovered in 1981. This AMP is an inducible antibacterial factor identified from the diapause pupa of silkworm, named cecropin. Currently, in the AMP database (https://aps.unmc.edu/AP/), there are about 325 insect-derived AMPs reported. These insect-derived AMPs can be divided into four categories according to their amino acid properties: (1) cysteine-containing AMPs; (2) α-helical AMPs; (3) proline-containing AMPs; and (4) glycine-containing AMPs.

Among many insects, Hermetia illucens larvae contain more than 50 AMP genes, and are the largest AMP-producing family found in insects so far. Meanwhile, it is discovered that Hermetia illucens exhibits an ability to transform wastes into its own resources, and can survive in a multi-pathogenic environment while producing corresponding antimicrobial substances. Hermetia illucens AMP has different expressions in different substrates, thus providing a scientific basis for the breeding of Hermetia illucens and the production of highly active AMPs. However, there is still a lack of a method for breeding the Hermetia illucens that produces highly active AMPs.

### SUMMARY

An objective of the present disclosure is to provide a medium for breeding a Hermetia illucens larva that produces a highly active AMP, a breeding method, and an extraction method of an AMP. In the present disclosure, the medium can realize the rapid and effective production of the highly active AMP by the Hermetia illucens larva.

The present disclosure provides a medium for breeding a Hermetia illucens larva that produces a highly active AMP, including wheat bran, where the medium has a moisture content of 65% to 70%.

The present disclosure further provides use of the medium in breeding a Hermetia illucens larva that produces a highly active AMP.

The present disclosure further provides a method for breeding a Hermetia illucens larva that produces a highly active AMP based on the medium, including the following steps:

breeding a Hermetia illucens larva at an age of 3 d in the medium, where the medium is turned over per day during the breeding.

Preferably, the breeding is conducted at a density of 1 larva/cm³.

The present disclosure provides an extraction method of a Hermetia illucens AMP, including the following steps:
collecting a Hermetia illucens larva bred by the method through sieving, injecting a microbial liquid into the larva to induce AMP production, conducting a starvation treatment for 24 h, and extracting the Hermetia illucens AMP by insect hemolymph collection.

Preferably, the collecting is conducted when the Hermetia illucens larva is at an age of 8 d.

Preferably, the sieving refers to separating the larva from the medium by an 8-mesh sieve.

Preferably, the microbial liquid includes a Staphylococcus aureus liquid; Staphylococcus aureus in the Staphylococcus aureus liquid has a concentration of 10⁶ cfu/μL to 10⁷ cfu/μL; and the microbial liquid is injected at 10 μL/per larva.

The present disclosure further provides a Hermetia illucens AMP extracted by the extraction method.

The present disclosure further provides use of the AMP in preparation of a microbial inoculant for inhibiting Staphylococcus aureus.

The present disclosure provides a medium for breeding a Hermetia illucens larva that produces a highly active AMP. In the present disclosure, the medium can realize the rapid and effective production of the highly active AMP by the Hermetia illucens larva. The test results show that in a pure wheat bran matrix with a moisture content of 70%, the Hermetia illucens larva is bred at a density of 1 larva/cm³ without adjusting a pH value and without adding salt or oil. Under these conditions, the larva producing a highly active AMP can be obtained. The AMP obtained based on the medium of the present disclosure can destroy a cell membrane structure of Staphylococcus aureus and change permeability of the cell membrane. The AMP can disrupt an integrity of S. aureus cells, leading to changes in cell surface morphology. The AMP can also cause cell lysis and cytoplasmic efflux of the S. aureus.

### BRIEF DESCRIPTION OF THE DRAWINGS

To describe the technical solutions in embodiments of the present disclosure or in the prior art more clearly, the accompanying drawings required in the embodiments are briefly described below. Apparently, the accompanying drawings in the following description show merely some embodiments of the present disclosure, and other drawings can be derived from these accompanying drawings by those of ordinary skill in the art without creative efforts.

FIG. 1 shows results of an inhibitory zone diameter (IZD) under different breeding conditions provided by the present disclosure;

FIGS. 2A-F show photos of an optical microscope and a fluorescence microscope of Staphylococcus aureus provided by the present disclosure; and

FIGS. 3A-F show photos of a scanning electron microscope (SEM) and a transmission electron microscope (TEM) of Staphylococcus aureus provided by the present disclosure.

### DETAILED DESCRIPTION OF THE EMBODIMENTS

The present disclosure provides a medium for breeding a Hermetia illucens larva that produces a highly active AMP, including wheat bran, where the medium has a moisture content of 65% to 70%. In the present disclosure, the moisture content is more preferably 70%. The wheat bran preferably includes flaked coarse wheat bran. The wheat bran has a particle size preferably on the order of microns to millimeters. The medium is used for the breeding of Hermetia illucens, and can relatively improve an antimicrobial ability of the Hermetia illucens AMP.

The present disclosure further provides use of the medium in breeding a Hermetia illucens larva that produces a highly active AMP.

The present disclosure further provides a method for breeding a Hermetia illucens larva that produces a highly active AMP based on the medium, including the following steps:

breeding a Hermetia illucens larva at an age of 3 d in the medium, where the medium is turned over per day during the breeding.

In the present disclosure, the 3-day-old Hermetia illucens is preferably obtained by hatching Hermetia illucens eggs. The hatching is conducted at preferably 25° C. to 30° C. The Hermetia illucens is preferably hatched on a Hermetia illucens feed. The breeding is conducted at a density of preferably 1 larva/cm³. The breeding is conducted at preferably 25° C. to 30° C.

The present disclosure provides an extraction method of a Hermetia illucens AMP, including the following steps:
collecting a Hermetia illucens larva bred by the method through sieving, injecting a microbial liquid into the larva to induce AMP production, conducting a starvation treatment for 24 h, and extracting the Hermetia illucens AMP by insect hemolymph collection. In the present disclosure, the collecting is conducted when the Hermetia illucens larva is at an age of 8 d. The sieving refers to preferably separating the larva from the medium by an 8-mesh sieve.

In the present disclosure, the microbial liquid includes preferably a Staphylococcus aureus liquid; Staphylococcus aureus in the Staphylococcus aureus liquid has a concentration of preferably 10⁶ cfu/μL to 10⁷ cfu/μL, more preferably 10⁶ cfu/μL; and the microbial liquid is injected at preferably 10 μL/per larva.

The present disclosure further provides a Hermetia illucens AMP extracted by the extraction method.

The present disclosure further provides use of the AMP in preparation of a microbial inoculant for inhibiting Staphylococcus aureus. The AMP can be used for the treatment of diseases caused by Staphylococcus aureus. A method of the treatment preferably includes applying the AMP of the present disclosure to patients caused by Staphylococcus aureus. The AMP can destroy a cell membrane structure of Staphylococcus aureus and change permeability of the cell membrane. The AMP can disrupt an integrity of S. aureus cells, leading to changes in cell surface morphology. The AMP can also cause cell lysis and cytoplasmic efflux of the S. aureus.

In order to further illustrate the present disclosure, the medium for breeding a Hermetia illucens larva that produces a highly active AMP, the breeding method, and the extraction method of an AMP provided by the present disclosure will be described in detail below in conjunction with accompanying drawings and examples, but they should not be construed as limiting the protection scope of the present disclosure.

### Example 1

1. Acquisition of feeding raw materials: pure wheat bran, a soybean cake powder, a peanut cake powder, and a rapeseed cake powder were dried in an oven at 60° C. to 70° C. for 3 h for later use, and then adjusted to a moisture content of 70% before feeding Hermetia illucens.

2. In an early stage, Hermetia illucens eggs were hatched with a small incubator. After the age in 3 d, the larvae were bred in separate boxes to an age of 8 d under different breeding conditions (as shown in (1) to (5)). During the breeding, the raw materials were turned over per day until 8-day-old larvae were obtained.

(1) A pH value of the pure wheat bran was separately adjusted to 3, 5, 7, 9, and 11, and were used to feed Hermetia illucens larvae (at a density of 0.5 larvae/cm³). At the age in 8 d, the larvae were separated from insect sand using an 8-mesh sieve, and a hundred of larvae were selected for later use.

(2) In breeding, pure wheat bran, pure wheat bran+soybean cake powder (1:1), pure wheat bran+peanut cake powder (1:1), and pure wheat bran+rapeseed cake powder (1:1) were used to feed Hermetia illucens larvae (at a density of 0.5 larvae/cm³). In the present disclosure, the above treatments were marked as A, B, C, and D, respectively. At the age in 8 d, the larvae were separated from insect sand using an 8-mesh sieve, and a hundred of larvae were selected for later use.

(3) A moisture content of the feed was adjusted to 50%, 60%, 70%, and 80%, respectively, and then used to feed Hermetia illucens larvae (the medium was pure wheat bran, and the density was 0.5 larvae/cm³). At the age in 8 d, the larvae were separated from insect sand using an 8-mesh sieve, and a hundred of larvae were selected for later use.

(4) The salt (0%, 1%, 2%, and 3%) or soybean oil (0%, 2%, 4%, and 6%) was added to the pure wheat bran separately, for feeding Hermetia illucens larvae. At the age in 8 d, the larvae were separated from insect sand using an 8-mesh sieve, and a hundred of larvae were selected for later use.

(5) A breeding density of larvae was adjusted to 0.25 larvae/cm³, 0.5 larvae/cm³, 0.75 larvae/cm³, 1 larva/cm³ separately, and then Hermetia illucens larvae were bred (the medium was pure wheat bran). At the age in 8 d, the larvae were separated from insect sand using an 8-mesh sieve, and a hundred of larvae were selected for later use.

3. The larvae isolated from (1) to (5) were rinsed, and induced with a microbial liquid of Staphylococcus aureus at a concentration of 10⁶ cfu/μL. That is, 10 μL of the microbial liquid was injected into the body of the Hermetia illucens larvae through a microsyringe.

4. After the induction, a starvation treatment was conducted for 24 h (the starvation treatment referred to placing the isolated Hermetia illucens larvae in a ventilated glass vessel without adding feed). The Hermetia illucens AMP was extracted by insect hemolymph collection, so as to obtain the Hermetia illucens larvae AMP.

5. IZD was determined by a filter paper method, and bacteriostatic activity and bacteriostatic rate were evaluated with the IZD, thereby determining optimal feed pH value, optimal feed ratio, optimal moisture content, optimal breeding density, and optimal addition of salt/soybean oil.

6. In order to further confirm that the Hermetia illucens AMP prepared under the optimal conditions had a desirable antimicrobial effect, the antimicrobial properties of the Hermetia illucens AMP were studied. The effects of AMP on the cell membrane and surface morphology of Staphylococcus aureus at concentrations of 36.97 μg/mL and 73.94 μg/mL (namely concentrations of MIC and 2MIC) were observed with optical electron microscope, fluorescence microscope, SEM, and TEM (the MIC concentrations were obtained by a two-fold dilution method).

### Experimental Results

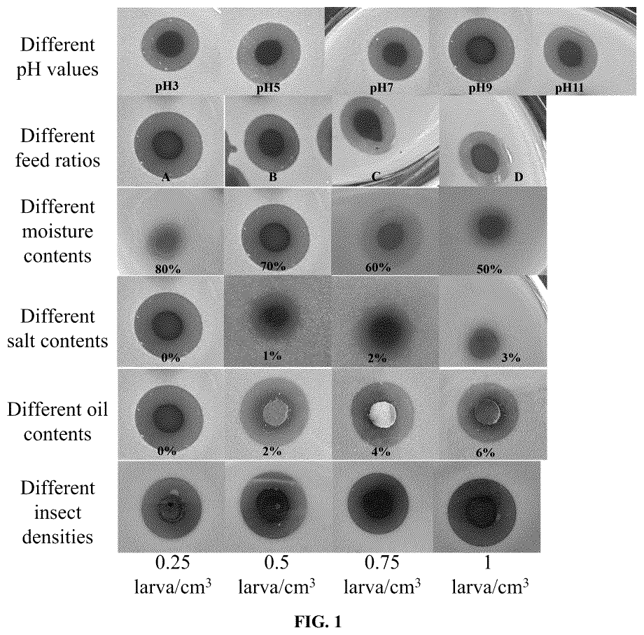

In this example, the antimicrobial activity of AMP produced by the Hermetia illucens was determined by IZD comparison. Test results were shown in Table 1 and FIG. 1. The results showed that after the induction of Hermetia illucens larvae bred under different pH conditions, it was found that pH did not affect the activity in antimicrobial activity of AMP produced by the Hermetia illucens. After induction of Hermetia illucens larvae bred with different feeding ratios, it was found that the activity in antimicrobial activity of AMP produced by the Hermetia illucens larvae bred with pure wheat bran was the best. After induction of Hermetia illucens larvae bred at different moisture contents, it was found that the activity in antimicrobial activity of AMP produced by the Hermetia illucens larvae bred at 70% moisture content was the best. After induction of Hermetia illucens larvae bred with different salt content, it was found that the activity in antimicrobial activity of AMP produced by the Hermetia illucens larvae bred without adding salt was the best. After induction of Hermetia illucens larvae bred with different oil contents, it was found that the activity in antimicrobial activity of AMP produced by the Hermetia illucens larvae bred without adding oil was the best (since there was no significant difference between 0% and 2% addition, in order to save costs, 0% addition was adopted). After induction of Hermetia illucens larvae cultured at different insect densities, it was found that the activity in antimicrobial activity of AMP produced by the Hermetia illucens larvae bred at 1 larva/cm³ was the best.

TABLE 1
IZD under different breeding conditions

| Treatment | IZD (mm) |
|---|---|
| Ph = 3 | 15.42 ± 0.1 a |
| pH = 5 | 16.87 ± 0.24 a |
| pH = 7 | 15.78 ± 0.93 a |
| pH = 9 | 16.76 ± 0.98 a |
| pH = 11 | 15.93 ± 0.19 a |
| Pure wheat bran | 16.76 ± 0.98 a |
| Soybean cake powder:wheat bran = 1:1 | 15.1 ± 0.57 b |
| Peanut cake powder:wheat bran = 1:1 | 14.2 ± 0.36 b |
| Rapeseed cake powder:wheat bran = 1:1 | 14.4 ± 0.31 b |
| Moisture content 50% | 13.99 ± 0.44 b |
| Moisture content 60% | 15 ± 0.13 b |
| Moisture content 70% | 16.76 ± 0.98 a |
| Moisture content 80% | 8.42 ± 0.56 c |
| Salt content 0% | 16.76 ± 0.98 a |
| Salt content 1% | 13.04 ± 0.37 b |
| Salt content 2% | 12.49 ± 0.41 b |
| Salt content 3% | 6.51 ± 0.16 c |
| Oil content 0% | 16.76 ± 0.98 a |
| Oil content 2% | 16.92 ± 0.61 a |
| Oil content 4% | 15.44 ± 0.82 ab |
| Oil content 6% | 14.86 ± 0.09 b |
| Insect density 0.25 larvae/cm³ | 16.68 ± 0.55 b |
| Insect density 0.5 larvae/cm³ | 17.04 ± 0.1 b |
| Insect density 0.75 larvae/cm³ | 16.89 ± 0.18 b |
| Insect density 1 larva/cm³ | 18.17 ± 0.07 a |

According to the above results, in the present disclosure, the optimal conditions were determined to be: in a pure wheat bran matrix with a moisture content of 70%, the Hermetia illucens larva was bred at a density of 1 larva/cm³ without adjusting a pH value and without adding salt or oil. Under these conditions, the larva producing a most highly active AMP could be obtained.

The characteristics of the Hermetia illucens larvae and the IZD data of AMP under optimal conditions were shown in Table 2:

Table 2 Characteristics of Hermetia illucens Larvae and IZD Data of AMP Under Optimal Conditions

| Average insect weight per larva | 0.064 | g |
|---|---|---|
| Crude protein content | 36.76% | |
| Crude fat content | 9.50% | |
| Moisture content | 79.97% | |
| IZD | 18.17 | mm |

The antimicrobial properties of AMP produced by Hermetia illucens larvae bred under optimal conditions were as follows:

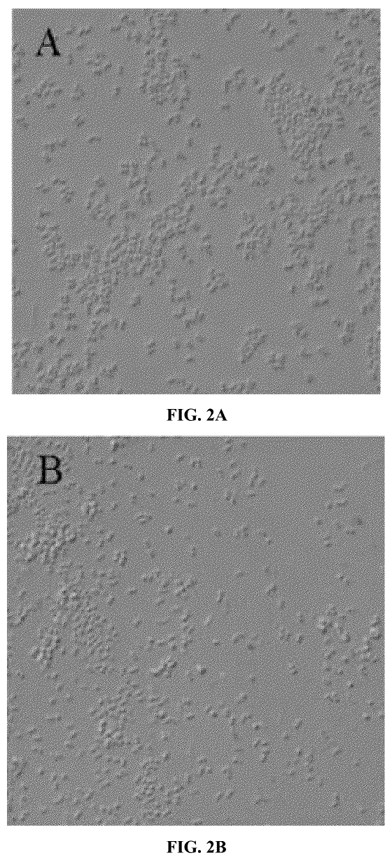

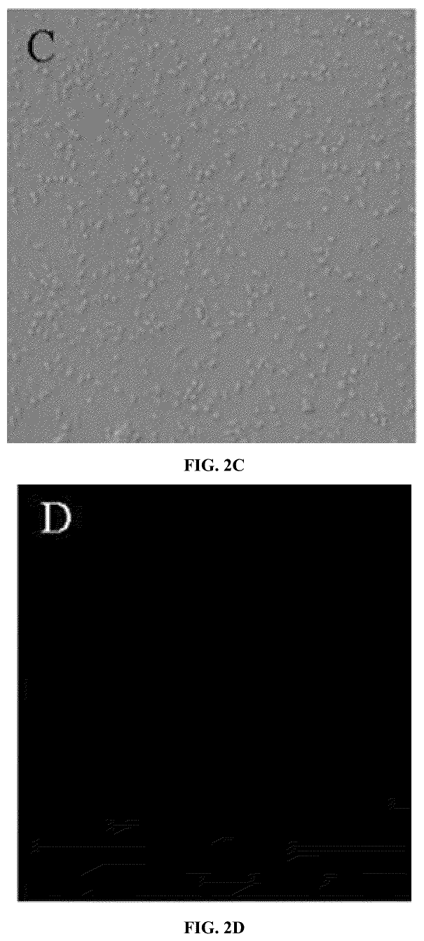

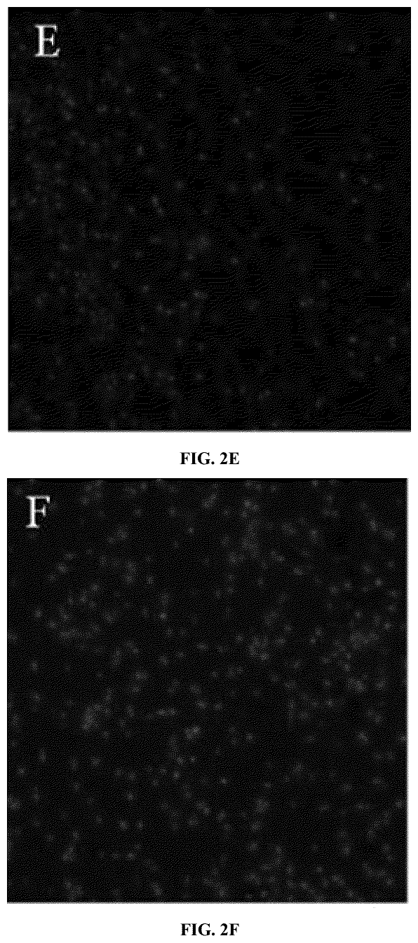

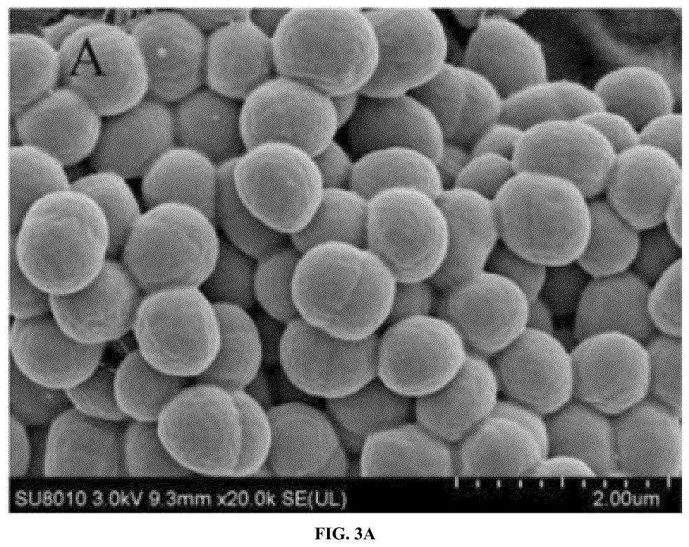

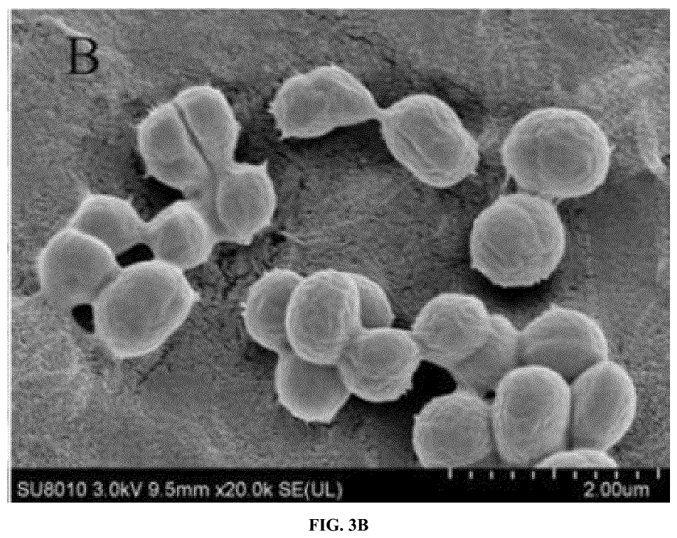

FIGS. 2A-F showed photos of an optical microscope and a fluorescence microscope of Staphylococcus aureus. FIG. 2A, FIG. 2B, and FIG. 2C were optical microscope photos. FIG. 2D, FIG. 2E, and FIG. 2F were fluorescence microscope photos. FIG. 2A and FIG. 2D were Staphylococcus aureus (control). FIG. 2B and FIG. 2E were AMP-treated Staphylococcus aureus at MIC. FIG. 2C and FIG. 2F were AMP-treated Staphylococcus aureus at 2MIC. The AMP destroyed the cell membrane, such that the PI dye entered the bacterial cell, resulting in red fluorescence.

Staphylococcus aureus treated with different concentrations of AMP could emit red fluorescence under green laser, indicating that the AMP could destroy the cell membrane structure of Staphylococcus aureus and change the permeability of the cell membrane. In this way, the PI dye entered the cell and combined with the nucleic acid, and red fluorescence appeared under the green laser.

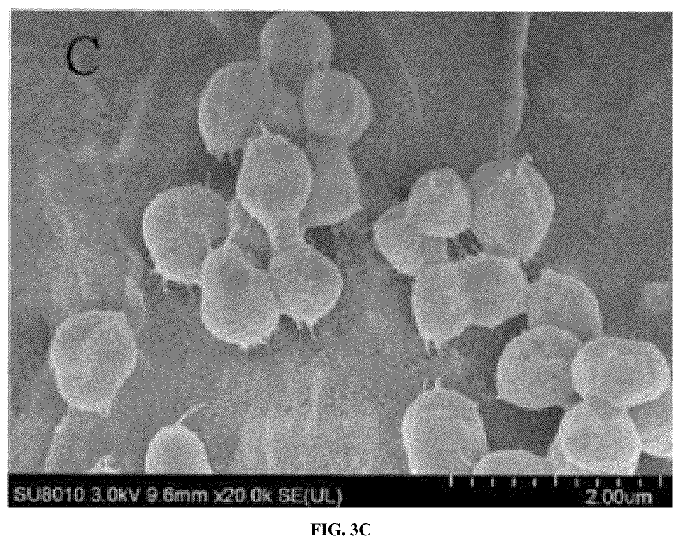

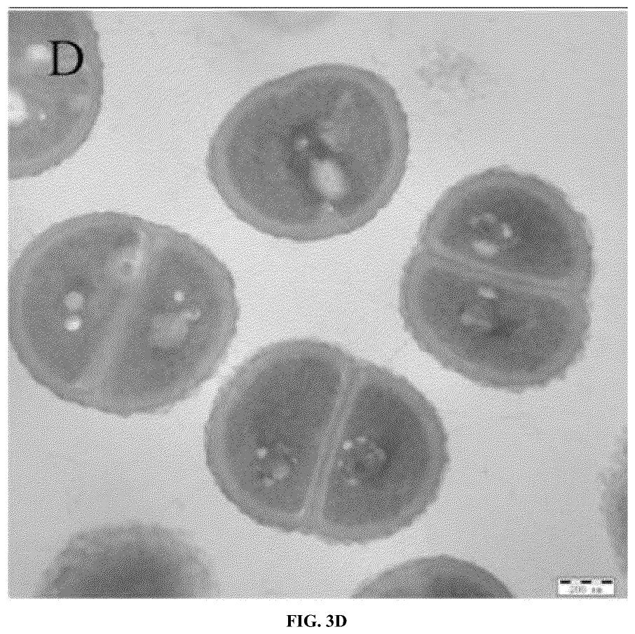

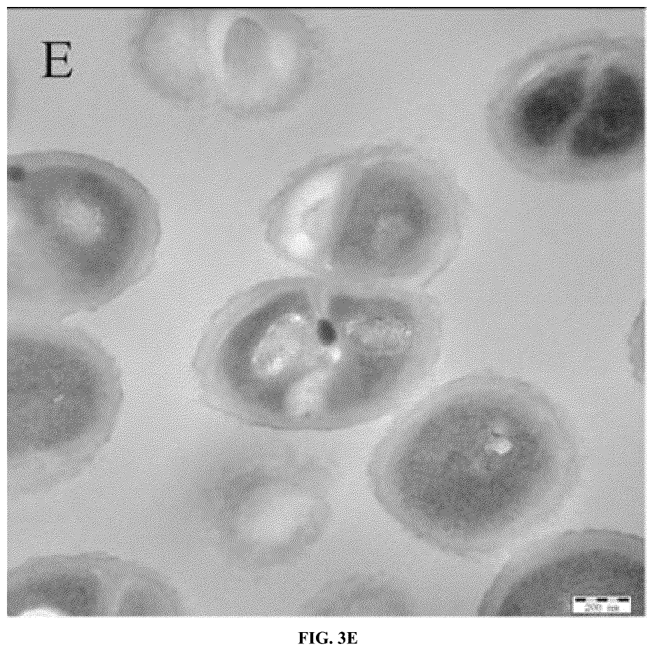

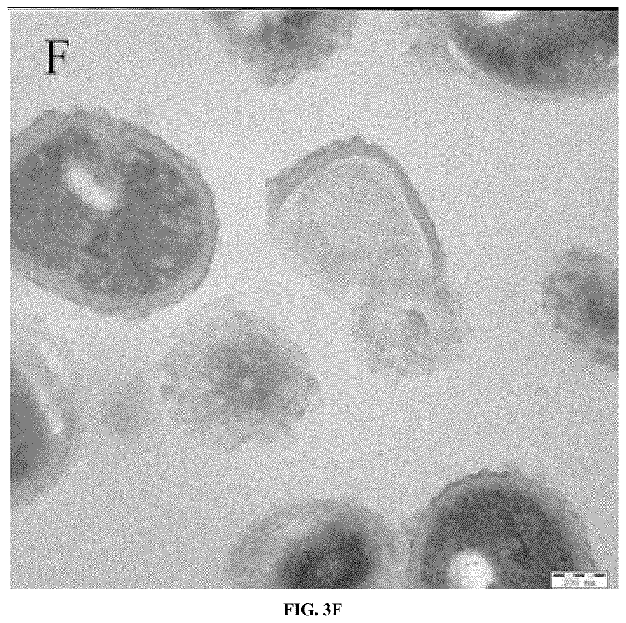

FIGS. 3A-F showed SEM and TEM images of Staphylococcus aureus. FIG. 3A, FIG. 3B and FIG. 3C were SEM images. FIG. 3D, FIG. 3E, and FIG. 3F were TEM images. FIG. 3A and FIG. 3D were original photo of Staphylococcus aureus (control). FIG. 3B and FIG. 3E were Staphylococcus aureus treated with AMP at MIC. FIG. 3C and FIG. 3F were Staphylococcus aureus treated with AMP at 2MIC.

The effect of AMP on the surface morphology of Staphylococcus aureus: it was observed by SEM that the surface of Staphylococcus aureus treated with AMP at MIC became wrinkled and cells adhered. These damages were more serious in cells treated at 2MIC. The results showed that the AMP was able to disrupt the cell integrity of S. aureus, resulting in changes in cell surface morphology.

The effect of AMP on intracellular microscopic changes of S. aureus: untreated S. aureus was observed to have a homogeneous cytoplasmic appearance with clear cell walls and membranes. However, some of the cell walls of Staphylococcus aureus treated with AMP were broken, and some cell debris could be seen in the surrounding environment of the cells. The results showed that the AMP could cause cell lysis and cytoplasmic efflux of S. aureus.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

What is claimed is:

1. A medium for breeding a Hermetia illucens larva that produces a highly active antimicrobial peptide (AMP), comprising wheat bran, wherein the medium has a moisture content of 65% to 70%.

2. A method for breeding a Hermetia illucens larva that produces a highly active AMP based on the medium according to claim 1, comprising the following steps:
breeding a Hermetia illucens larva at an age of 3 d in the medium according to claim 1, wherein the medium is turned over per day during the breeding.

3. The method according to claim 2, wherein the breeding is conducted at a density of 1 larva/cm³.

4. An extraction method of a Hermetia illucens AMP, comprising the following steps:
collecting a Hermetia illucens larva bred by the method according to claim 2 through sieving, injecting a microbial liquid into the larva to induce AMP production, conducting a starvation treatment for 24 h, and extracting the Hermetia illucens AMP by insect hemolymph collection.

5. The extraction method according to claim 4, wherein the breeding is conducted at a density of 1 larva/cm³.

6. The extraction method according to claim 4, wherein the collecting is conducted when the Hermetia illucens larva is at an age of 8 d.

7. The extraction method according to claim 5, wherein the collecting is conducted when the Hermetia illucens larva is at an age of 8 d.

8. The extraction method according to claim 4, wherein the sieving refers to separating the larva from the medium by an 8-mesh sieve.

9. The extraction method according to claim 5, wherein the sieving refers to separating the larva from the medium by an 8-mesh sieve.

10. The extraction method according to claim 4, wherein the microbial liquid comprises a Staphylococcus aureus liquid; Staphylococcus aureus in the Staphylococcus aureus liquid has a concentration of 10⁶ cfu/μL to 10⁷ cfu/μL; and the microbial liquid is injected at 10 μL/per larva.

11. The extraction method according to claim 5, wherein the microbial liquid comprises a Staphylococcus aureus liquid; Staphylococcus aureus in the Staphylococcus aureus liquid has a concentration of 10⁶ cfu/μL to 10⁷ cfu/μL; and the microbial liquid is injected at 10 μL/per larva.

12. A Hermetia illucens AMP extracted by the extraction method according to claim 4.

13. The Hermetia illucens AMP according to claim 12, wherein the breeding is conducted at a density of 1 larva/cm³.

14. The Hermetia illucens AMP according to claim 12, wherein the collecting is conducted when the Hermetia illucens larva is at an age of 8 d.

15. The Hermetia illucens AMP according to claim 12, wherein the sieving refers to separating the larva from the medium by an 8-mesh sieve.

16. The Hermetia illucens AMP according to claim 13, wherein the sieving refers to separating the larva from the medium by an 8-mesh sieve.

17. The Hermetia illucens AMP according to claim 12, wherein the microbial liquid comprises a Staphylococcus aureus liquid; Staphylococcus aureus in the Staphylococcus aureus liquid has a concentration of 10⁶ cfu/μL to 10⁷ cfu/μL; and the microbial liquid is injected at 10 μL/per larva.

18. A preparation method of a microbial inoculant for inhibiting Staphylococcus aureus by using the AMP according to claim 12.

19. The preparation method according to claim 18, wherein the breeding is conducted at a density of 1 larva/cm³.

20. The preparation method according to claim 18, wherein the collecting is conducted when the Hermetia illucens larva is at an age of 8 d.
